(19) United States
(12) Patent Application Publication
(10) Pub. No.: US 2026/0226397 A1
(43) Pub. Date: Aug. 6, 2026

(54) METHOD AND SYSTEM FOR TREATING BIOMASS MATERIAL

(71) Applicant: VALMET AB, Sundsvall (SE)

(72) Inventor: Francois LAMBERT, Sundsvall (SE)

(73) Assignee: VALMET AB, Sundsvall (SE)

(21) Appl. No.: 19/156,064

(22) PCT Filed: Feb. 6, 2024
(86) PCT No.: PCT/SE2024/050101

§ 371 Date: Aug. 13, 2025

(30) Foreign Application Priority Data
Feb. 14, 2023 (SE) 2330081-7

(51) Int. Cl. C12M 1/00; D21C 1/04; D21C 3/06; D21C 7/12

(52) U.S. Cl. CPC C12M 45/06; D21C 1/04; D21C 3/06; D21C 7/12

(57) ABSTRACT

Method for treating biomass material, comprising impregnating (201) the biomass material with sulfur dioxide and/or sulfurous acid in an impregnation reactor, subjecting the biomass material to hydrothermal treatment (202) in a pretreatment reactor, and determining (204) a flow, or a parameter indicative of a flow, of gases vented off from an upper portion of the pretreatment reactor The method further comprises controlling (205a, 205b) the impregnating based on the flow of gases, or the parameter indicative of the flow of gases. A corresponding system is also provided.

### TECHNICAL FIELD

The invention relates to the field of treating biomass material such as lignocellulose biomass material, which treating comprises impregnating the biomass material with sulfur dioxide and/or sulfurous acid followed by hydrothermal treatment in a pressurized pretreatment reactor.

### BACKGROUND

Methods for treating biomass material such as lignocellulose biomass material, which treating comprises impregnating the biomass material with sulfur dioxide and/or sulfurous acid followed by hydrothermal treatment in a pressurized pretreatment reactor are known in the art.

The biomass material may be impregnated with sulfur dioxide in gaseous form or with an impregnation liquid comprising an impregnation liquid comprising sulfur dioxide absorbed in water and/or sulfurous acid. Sulfur dioxide absorbed in water is sometimes referred to as sulfurous acid solution.

WO2020093131 discloses a process for converting softwood to glucose or a biofuel, where the softwood is subject to a pretreatment with sulfur dioxide and/or bisulfite prior to enzymatic hydrolysis. The sulfur dioxide charge (concentration) is determined as a specific concentration of the impregnation liquid or based on the dry solid content of the feedstock. WO2020093131 discloses that high sulfur dioxide charges are advantageous for the following enzymatic hydrolysis stage.

### SUMMARY

An object of the invention is to provide an improved method for treating comprising impregnating the biomass material with sulfur dioxide and/or sulfurous acid followed by hydrothermal treatment/pretreatment in a pressurized reactor. A further object is to achieve improved control of the impregnation of the biomass material.

These and other objects are achieved by the present invention by means of a method and a system according to the independent claims.

According to a first aspect of the invention, a method for treating biomass material such as lignocellulose material is provided. The method comprises impregnating the biomass material with sulfur dioxide and/or sulfurous acid (for example in an impregnation reactor) and subjecting the biomass material to hydrothermal treatment in a pretreatment reactor. The method further comprises determining a flow, or a parameter indicative of a flow, of gases vented off from an upper portion of the pretreatment reactor, and controlling said impregnating based on said flow of gases, and/or based on said parameter indicative of said flow.

In other words, the method comprises impregnating the biomass material with sulfur dioxide and/or sulfurous acid, subjecting the impregnated biomass material to hydrothermal treatment in a pretreatment reactor (by means of adding steam to the pretreatment reactor), and determining a flow of gases vented off from an upper portion of the pretreatment reactor (or a parameter indicative of the flow). The method further comprises controlling the impregnating (i.e. the conditions during the impregnating) based on the determined flow of gases, or parameter indicative of the flow of gases. Normally, the hydrothermal treatment/pretreatment is carried out in a separate pretreatment reactor, i.e. the method comprises feeding the impregnated biomass material from the impregnation reactor to the pretreatment reactor, i.e. the hydrothermal treatment is carried out after the impregnation. It is however foreseeable within the scope of the invention that the impregnating and the pretreatment is carried out in a common reactor, i.e. biomass material, impregnation liquid and steam is added to the same common reactor (a pretreatment reactor).

The flow, or a parameter indicative of a flow, may be determined by determining a valve opening of a valve through which said gases vented off from an upper portion of the pretreatment reactor pass. Alternatively, or additionally, determining the flow, or a parameter indicative of a flow, may comprise using a flow meter.

It is understood that hydrothermal treatment may also be referred to as pretreatment, or more specifically pre-hydrolysis. Pre-hydrolysis is carried out at milder conditions compared to a hydrolysis process and is used for hydrolyzing the hemicellulose content of the biomass. The above-described determining of a parameter indicative of the flow may be based on valve opening parameter(s) (such as a signal/voltage from a position sensor coupled to the valve) or flow measuring device parameter(s) (such as a signal/voltage from the flow measuring device), which may both be described as parameter(s) or signal(s) indicative of the flow. It is understood that the determining based on such parameter(s)/signals(s) does not necessarily need to result in a flow parameter in the sense of being defined in for example m3/h, but may on the contrary for example use signals/voltages directly to result in a parameter indicative of the flow. Depending on the system used to carry out the method, different parameters indicative of said flow may be identified and used in the method.

The invention is based on the insight that the concentration of sulfur dioxide/sulfurous acid in the biomass material/liquid phase is dependent on the sulfur dioxide partial pressure in the pretreatment reactor. The invention is further based on the insight that sulfur dioxide partial pressure is dependent on the venting/degassing from the pretreatment reactor. Consequently, there is a correlation between the concentration of sulfur dioxide/sulfurous acid in the liquid phase/biomass material and the venting/degassing. The inventors have consequently realized that the charge of sulfur dioxide/sulfurous acid added to the material in the impregnation stage is advantageously controlled depending on the venting/degassing flow from the reactor. For example, if the venting increases, the charge should be decreased, and if the venting decreases, the charge should be increased.

The biomass material may for example be of woody origin, for example wood chips or flakes, bark, sawdust or agricultural residues such as straw, bagasse or empty fruit bunches.

In embodiments, the step of controlling said impregnating comprises:
controlling a charge of sulfur dioxide and/or sulfurous acid during said impregnating based on said flow of gases, or said parameter indicative of said flow, and/or
controlling a residence time in said impregnation reactor based on said flow of gases, or said parameter indicative of said flow, and/or
controlling venting from the impregnation reactor based on said flow of gases, or said parameter indicative of said flow based on said flow of gases, or said parameter indicative of said flow.

In embodiments, the impregnating comprises adding sulfur dioxide and/or sulfurous acid to the impregnation reactor in the form of an impregnation liquid comprising sulfur dioxide absorbed in water and/or sulfurous acid. Aqueous solutions of sulfur dioxide at equilibrium and at a pH used for impregnation mainly comprises sulfur dioxide in the form of sulfur dioxide and bisulfite ion (HSO₃⁻), but also to some extent sulfurous acid and sulfite ion (SO₃²⁻). Such aqueous solutions may be described as comprising sulfur dioxide dissolved and/or sulfurous acid or simply as sulfurous acid solution.

In embodiments where such impregnation liquid is added, the method comprises controlling the hydrothermal treatment such that the temperature and pressure in the pretreatment reactor is constant (or such that the temperature and the sulfur dioxide partial pressure in the pretreatment reactor is constant), wherein the step of controlling the impregnating comprises:
if said flow, or said parameter indicative of said flow, increases, reducing the amount of impregnation liquid added to the impregnation reactor and/or decreasing the concentration of the impregnation liquid added to the impregnation reactor and/or decreasing a residence time in the impregnation reactor, and
if said flow, or said parameter indicative of said flow, decreases, increasing the amount of impregnation liquid added to the impregnation reactor and/or increasing the concentration of the impregnation liquid added to the impregnation reactor and/or increasing a residence time in the impregnation reactor.

In other embodiments, the impregnating comprises adding sulfur dioxide in gaseous form.

In embodiments where sulfur dioxide is added in gaseous form, the method comprises controlling the hydrothermal treatment such that temperature and pressure in the pretreatment reactor is constant (or such that the temperature and the sulfur dioxide partial pressure in the pretreatment reactor is constant), wherein the step of controlling the impregnating comprises:
if said flow, or said parameter indicative of said flow, increases, decreasing a pressure in the impregnation reactor and/or decreasing a residence time in the impregnation reactor and/or increasing venting from the impregnation reactor, and
if said, or said parameter indicative of said flow, flow decreases, increasing a pressure in the impregnation reactor and/or increasing a residence time in the impregnation reactor and/or increasing venting from the impregnation reactor.

The above-mentioned control of the hydrothermal treatment such that temperature and pressure (or SO2 partial pressure) in the pretreatment reactor is constant may comprise controlling an amount of steam fed to the pretreatment reactor and/or controlling a flow of gases vented off from an upper portion of the pretreatment reactor. It is understood that the pretreatment reactor may be fed steam at one, two or more positions, and that at least one, or each, of the flows of steam may be controlled to achieve the constant temperature and pressure. Such control is advantageous since it allows the temperature and pressure to be quickly controlled.

In embodiments, the method further comprises, prior to impregnating, compressing the biomass material, whereafter the biomass material is fed into an inlet of the impregnation reactor such that it expands into the impregnation reactor.

The sulfur dioxide partial pressure in the pretreatment reactor may be estimated as follows:
measuring the temperature and pressure in the reactor,
calculating the steam saturation pressure at the measured temperature,
estimating the sulfur dioxide partial pressure by subtracting the steam saturation pressure at said temperature from the pressure.

The impregnation liquid may be added/injected into the impregnation reactor such that it is absorbed by the biomass material and/or such that an impregnation liquid level is formed in said impregnation reactor, which impregnation liquid level is (vertically) below said inlet. Impregnation liquid is added into the impregnation reactor in such a manner that it is (immediately) absorbed by the biomass material, i.e. at a flow and at such a position that substantially all of the injected impregnation liquid is absorbed by the biomass material. Alternatively, the impregnation liquid is added into the impregnation reactor in such a manner that (some of the) impregnation liquid is absorbed by the biomass material and (some of the) impregnation liquid is accumulated to form an impregnation liquid level, which level is (vertically) below the biomass inlet, however. Consequently, impregnation liquid is added to the impregnation reactor, but the biomass material is not soaked in impregnation liquid, nor is it fed into impregnation liquid (since the optional liquid level is below the inlet). This may be particularly advantageous for biomass material such as saw dust having a high dry content, for instance at least 60% or 70% or 80% or 90%, since such biomass material may be difficult to handle/convey if being fed into the impregnation below a liquid level.

Alternatively, the impregnation reactor is at least partly filled with impregnation liquid up to a liquid level, wherein said feeding comprises feeding the biomass material being into said impregnation reactor at a lower level than said liquid level. This is advantageous since the biomass material may be kept soaked in the impregnation liquid for the whole, or at least a large portion of, the residence time in the impregnation reactor.

In embodiments where the impregnation reactor is arranged substantially vertically and/or at an acute angle relative a vertical direction, the method further comprises conveying the biomass material upwards in the impregnation reactor towards an outlet at an upper portion of the impregnation reactor where the biomass material is discharged and fed to the pretreatment reactor. Such upward conveying may be achieved for example by means of a conveyor screw, and the residence time in the impregnation reactor may thus be controlled by controlling the speed of the conveyor screw.

In embodiments, a concentration of the impregnation liquid is within an interval corresponding to 28-165 g sulfur dioxide/l, or 40-113 g sulfur dioxide/l, or 56-79 g sulfur dioxide/l. Such a concentration may be referred to as a “sulfur dioxide concentration” but takes into account not only the actual sulfur dioxide content, but also the concentrations of bisulfite ion and sulfurous acid (and other compounds formed from sulfur dioxide, if present) on a molar basis converted into a corresponding concentration of sulfur dioxide.

In embodiments, the method comprises discharging the material from the pretreatment reactor using a steam explosion discharge device which comprises a blow valve/discharge nozzle through which the biomass material is discharged while undergoing a rapid pressure reduction, for instance to atmospheric pressure. In embodiments, the method further comprises separating the blow steam (which is discharged from the blow valve along with the biomass) from the biomass with a separator, for example a cyclone or a centrifuge. Alternatively, the biomass material may be discharged into a blow tank from which blow steam is vented off. Blow steam in this context refers to a mixture of (water) steam and volatile organic compounds released from the biomass material during thermal treatment/steam explosion discharge of the biomass material. The blow steam also comprises sulfur dioxide which means that the blow steam is advantageously recycled to recover the sulfur dioxide for use as part of the impregnation liquid.

In embodiments, the method further comprises subjecting the steam exploded biomass to enzymatic hydrolysis for hydrolyzing the cellulose to sugars, and thereafter an optional fermentation stage. Enzymatic hydrolysis and fermentation stages are well known in the art and will not be described in further detail here.

According to a second aspect of the invention, a system for treating biomass material such as lignocellulose biomass material is provided. The system comprises an impregnation reactor, a pretreatment reactor and a control system. The impregnation reactor is arranged to receive the biomass material, the impregnation reactor comprising means for adding sulfur dioxide and/or sulfurous acid for impregnation of the biomass material. The pretreatment reactor is arranged to receive impregnated biomass material from the impregnation reactor for hydrothermal treatment, the pretreatment reactor being provided with measuring means for determining a flow, or a parameter indicative of a flow, of gases emitted from an upper portion of the pretreatment reactor. The control system is configured to control the impregnation reactor based on said flow, or said parameter indicative of said flow, of gases. It is understood that the pretreatment reactor comprises one or more steam injection means for adding steam into the reactor for heating of the biomass material to achieve the hydrothermal treatment.

In embodiments, the control system is configured to control the means for adding sulfur dioxide and/or sulfurous acid based on the flow of gases or based on the parameter indicative of said flow of gases.

In embodiments, the impregnation reactor is furthermore provided with means for conveying the biomass material through the reactor and/or at least one controllable valve arranged at an upper portion of the impregnation reactor to allow gases to be vented off from the reactor, wherein the control system is configured to control said means for adding sulfur dioxide and/or sulfurous acid and/or said means for conveying and/or said controllable valve based on said flow, or said parameter indicative of said flow, of gases.

The above-described methods and systems are advantageously continuous, i.e. the steps of the methods are carried out continuously and the devices of the systems are configured to operate continuously.

The features of the embodiments described above are combinable in any practically realizable way to form embodiments having combinations of these features. Further, all features and advantages of embodiments described above with reference to the first aspect of the invention may be applied in corresponding embodiments of the system according to the second aspect of the invention and vice versa.

### BRIEF DESCRIPTION OF THE DRAWINGS

Above discussed and other aspects of the present invention will now be described in more detail using the appended drawings, which show presently preferred embodiments of the invention, wherein:

FIG. 1 shows a schematic illustration of an embodiment of the system according to the second aspect of the invention;

FIG. 2 shows a flow chart of an embodiment of the method according to the first aspect of the invention;

FIG. 3 shows a flow chart of another embodiment of the method according to the first aspect of the invention.

### DETAILED DESCRIPTION

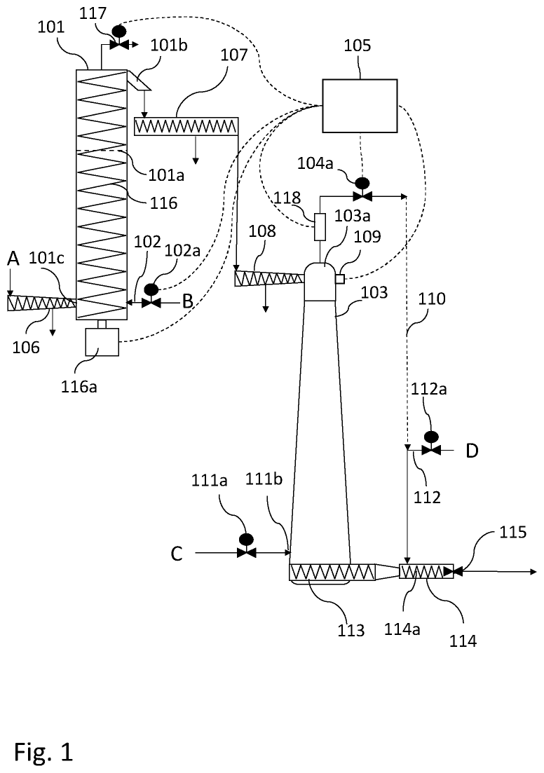

FIG. 1 shows a schematic illustration of an embodiment of the system according to the second aspect of the invention.

Raw material A (lignocellulose biomass material, for example wood, bark, bagasse or straw) is fed to the plug screw feeder 106 for compressing and transporting the biomass material into the impregnation reactor/tower 101 which is substantially vertically arranged. The plug screw feeder compresses the biomass material to a tight plug which expands when it leaves the tapered screw and enters impregnation reactor 101 at inlet 101c. The material expansion will cause a sponge effect and sulfur dioxide and/or sulfurous acid is rapidly sucked into the porous structure of the biomass.

The impregnation reactor 101 comprises an inlet 102 for adding an impregnation liquid B comprising sulfurous acid and/or sulfur dioxide absorbed in water. The flow of impregnation liquid is controllable using electrically controllable valve 102a. As can be seen in FIG. 1, the inlet 102 is vertically aligned with inlet 101c/plug screw feeder 106 such that the impregnation liquid is injected towards the biomass material inlet i.e. onto the biomass material while expanding into the reactor 101. This could be described as the impregnation liquid being “sprayed” onto the biomass material (although inlet 102 is normally not a spray nozzle).

Optionally, the impregnation reactor 101 is partly filled with impregnation liquid up to a liquid level 101a, which liquid level is above the inlet 101c such that the biomass material is fed into impregnation liquid while undergoing expansion.

Typical operating conditions for the impregnation reactor are as follows:
Concentration of sulfurous acid and/or sulphur dioxide in the impregnation liquid corresponds to 28-165 g sulphur dioxide/l, preferably 40-113 g sulphur dioxide/l, or 56-79 g sulphur dioxide/l.
Mass of sulphur dioxide in the biomass material after impregnation 1-100 kg SO₂/metric ton dry biomass material, preferably 5-30 kg SO₂/metric ton dry biomass material, or 8-20 kg SO₂/metric ton dry biomass material.
Impregnation liquid temperature 5-60° C., preferably 20-50° C. or 30-40° C.
Residence time in the impregnation reactor is 20 s-60 minutes, preferably 20 s-30 minutes or 20 s-5 minutes or 20 s-50 s.

An optional electrically controllable valve 117 is connected via a conduit to a top portion of the impregnation reactor 101 such as to allow control of degassing from the impregnation reactor for pressure control.

The impregnation reactor is further provided with a conveyor screw 116 arranged to transport the biomass material upwards in the impregnation reactor towards an outlet 101b at an upper portion of the impregnation reactor where the biomass material is discharged to a transport screw 107 for transporting the biomass to a second plug screw feeder 108 which in turn feeds a pressurized pretreatment reactor 103. The conical plug screw feeder 108 compresses the biomass to a gas-tight plug which seals the pressure of the reactor 103 to the atmospheric. The plug screw feeder 108 may optionally be replaced with other technical solutions such as a rotary lock feeder or a lock hopper system. The pretreatment reactor is substantially vertically arranged. Biomass from the plug screw 108 falls by gravity inside the reactor 103 and piles up inside the reactor. The biomass pile slowly moves downwards as it is continuously emptied in the bottom of the reactor 103 with a discharge screw 113. Steam C is added to a lower portion of the reactor 103 by means of steam injection nozzle 111b. The steam flow is controlled by valve 111a which may be electrically controllable. The steam heats the biomass inside the reactor 103 such that the biomass is subjected to hydrothermal treatment (pre-hydrolysis) at elevated pressure and temperature. As indicated in FIG. 1, dewatering of the biomass takes place in screws 106-108.

A discharge device is formed by discharge screw 113 arranged at the bottom portion of the reactor and a thereto connected steam explosion device 114, 114a. The discharge screw 113 continuously empties the bottom of the reactor. The discharge screw is of the pressure-sealing type, i.e. is gas-tight just like the plug screw feeder 108 which means that no steam passes concurrently with biomass to the discharge chamber 114 of the steam explosion device, which further comprises a blow valve/discharge nozzle 115. The steam explosion device further comprises a transport screw 114a arranged in the discharge chamber to convey the biomass towards the blow valve/discharge nozzle 115.

Typical operating conditions for the pretreatment reactor are as follows:
Temperature in reactor: 160-225° C., preferably 190-210° C.
Pressure: corresponding pressure 5-30 bar(g)
Residence time: 1 min-3 hours, preferably 3-30 minutes
Delta pressure at discharge nozzle: 2-30 bar, preferably 2-15 bar or 2-10 bar.

Accumulated VOC/NCG, excess steam and SO₂ in the gas phase of reactor 103 are vented off (“degassed”) from the top portion 103a of the reactor. An electrically controllable valve 104 (also referred to herein as a controllable degassing device) is provided to allow control of the vented off gases.

An optional flow measuring device 118 is provided in the venting off conduit connected to the top portion 103a of the pretreatment reactor 103. As can be seen in FIG. 1, the flow measuring device 118 is electrically connected to the control system 105 to provide a signal thereto (such as a signal indicating a flow therethrough).

Optionally, the vented off gases are led to the discharge chamber 114 via a conduit 110 connecting the valve 104 with the discharge chamber 114.

A steam injection device 112b is provided which is connected to discharge chamber 114 to provide (fresh non-recirculated) discharge steam D to the discharge chamber 107a for control of the steam explosion discharge over discharge nozzle 115.

A pressure and temperature sensor 109 arranged at the top portion 103a of the vessel. The valves 102a, 104a and sensor 109 are electrically connected to a control system 105.

The control system 105 is optionally configured to calculate a steam saturation pressure based on pressure and temperature in the reactor 103 measured by sensor 109, and to estimate the sulfur dioxide partial pressure by the subtracting the steam saturation pressure from the pressure measured by sensor 109.

The electrically controllable valve 104a is optionally also configured to provide a feedback signal to the control system 105 indicating a current valve opening. Control system 105 is also connected to valve 111a and/or to valve 112a to control the steam flow to the pretreatment reactor and/or discharge chamber 114.

Optionally, the control system 105 is further connected to motor 116a driving the conveyor screw 116 to control the residence time in the impregnation reactor 101. Optionally, the control system 105 is further connected to electrically controllable valve 117 to control the pressure in the impregnation reactor 101.

The control system 105 is configured to determine a flow, or a parameter indicative of a flow, of gases emitted from the upper portion 103a of the pretreatment reactor 103 using signals received from valve 104a and/or flow measuring device 118.

The control system 105 is further configured to:
control valve 111a and/or valve 104a such that temperature and pressure in the pretreatment reactor 103 is constant (or such that the temperature and the sulfur dioxide partial pressure in the pretreatment reactor is constant);
if the determined flow, or the determined parameter indicative of the flow, increases, control valve 102a to reduce the amount of impregnation liquid added to the impregnation reactor and/or control motor 116a to decrease a residence time in the impregnation reactor, and
if the determined flow, or determined parameter indicative of the flow, decreases, control valve 102 to increase the amount of impregnation liquid added to the impregnation reactor and/or control motor 116a to increase a residence time in the impregnation reactor.

Steam exploded biomass material is discharged from discharge nozzle 115 along with blow steam to a blow tank (not shown in the figure) from which the blow steam is vented off. The blow steam may optionally be fed to an optional SO₂ recovery system (not shown in the figure) for recycling of SO₂ to the impregnation reactor 101.

The system may further comprise an enzymatic hydrolysis stage (not shown in the figure) arranged to receive biomass material from the blow tank, and optionally also a fermentation stage (not shown in the figure) arranged to receive material comprising sugars from the hydrolysis stage for fermentation of the sugars.

An alternative embodiment corresponds to the above-described embodiment in FIG. 1 except that impregnation reactor 101 is replaced with an impregnation reactor provided with one or more inlets for adding sulfur dioxide in gaseous form rather than the inlet 102 for adding impregnation liquid, i.e. the biomass material is impregnated with sulfur dioxide in gaseous form in this alternative embodiment.

In such an alternative embodiment, the control system is alternatively configured to
control valve 111a and/or valve 104a such that temperature and pressure in the pretreatment reactor 103 is constant (or such that the temperature and the sulfur dioxide partial pressure in the pretreatment reactor is constant);
if the determined flow, or the determined parameter indicative of the flow, increases, control valve 102a and/or 117 to decrease a pressure in the impregnation reactor and/or control motor 116a to decrease a residence time in the impregnation reactor and/or control valve 117 to decrease venting from the impregnation reactor, and
if the determined flow, or the determined parameter indicative of the flow, decreases, control valve 102a and/or 117 to increase a pressure in the impregnation reactor and/or control motor 116a to increase a residence time in the impregnation reactor and/or control valve 117 to increase venting from the impregnation reactor.

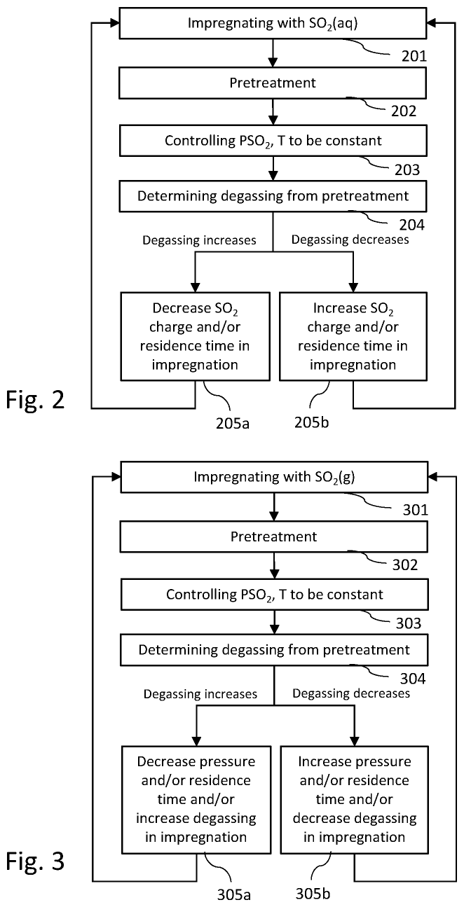

FIG. 2 shows a flow chart of an embodiment of the method according to the first aspect of the invention. The method comprises impregnating 201 the lignocellulose biomass material by adding sulfur dioxide and/or sulfurous acid to the impregnation reactor in the form of an impregnation liquid comprising sulfur dioxide absorbed in water and/or sulfurous acid. After the impregnating, the biomass material and steam are fed to a pretreatment reactor for pretreatment/hydrothermal treatment 202 of the biomass material. The flow of steam to the pretreatment reactor and/or a flow of gases vented off from the pretreatment reactor is/are controlled 203 such that temperature and S02 partial pressure (or total pressure) in the pretreatment reactor is constant. A flow, or a parameter indicative of a flow, of gases vented off (“degassing”) from an upper portion of the pretreatment reactor is determined 204. The impregnation is controlled based on the flow of gases, or the parameter indicative of the flow as follows:
if the flow, or the parameter indicative of the flow, increases, the SO2 charge is decreased by reducing the amount of impregnation liquid added to the impregnation reactor and/or decreasing the concentration of the impregnation liquid added to the impregnation reactor, and/or the residence time in the impregnation reactor is decreased (step 205a), and
if the flow, or the parameter indicative of the flow, decreases, the SO2 charge is increased by increasing the amount of impregnation liquid added to the impregnation reactor and/or increasing the concentration of the impregnation liquid added to the impregnation reactor, and/or the residence time in the impregnation reactor is increased (step 205b).

FIG. 3 shows a flow chart of another embodiment of the method according to the first aspect of the invention. The method comprises impregnating 301 the lignocellulose biomass material by adding sulfur dioxide in gas phase to the impregnation reactor. After the impregnating, the biomass material and steam are fed to a pretreatment reactor for pretreatment/hydrothermal treatment 302 of the biomass material. The flow of steam to the pretreatment reactor and/or a flow of gases vented off from the pretreatment reactor is/are controlled 303 such that temperature and pressure SO₂ partial pressure (or total pressure) in the pretreatment reactor is constant. A flow, or a parameter indicative of a flow, of gases vented off (“degassing”) from an upper portion of the pretreatment reactor is determined 304. The impregnation is controlled based on the flow of gases, or the parameter indicative of the flow as follows:
if the flow, or the parameter indicative of the flow, increases, the pressure in the impregnation reactor is decreased and/or a residence time in the impregnation reactor is decreased and/or venting from the impregnation reactor is decreased (step 305a), and
if the flow, or the parameter indicative of the flow, decreases, the pressure in the impregnation reactor is increased and/or a residence time in the impregnation reactor is increased and/or venting from the impregnation reactor is increased (step 305b).

The above-described embodiments are continuous, i.e. the method steps are carried out continuously and the devices of the systems are configured to operate continuously.

The description above and the appended drawings are to be considered as non-limiting examples of the invention. The person skilled in the art realizes that several changes and modifications may be made within the scope of the invention. For example, the reactors do not need to be vertically arranged. As a further example, the number of inlets for impregnation liquid and steam and the placement thereof can be varied. Further, the determining of a flow of gases vented off from the pretreatment reactor can be carried out using other means than the shown flow measuring device and/or electrically controllable valve.

1. A method for treating biomass material, comprising:
impregnating the biomass material with sulfur dioxide and/or sulfurous acid in an impregnation reactor;
after said impregnating, subjecting the biomass material to hydrothermal treatment in a pretreatment reactor; and
determining a flow of gases vented off from an upper portion of the pretreatment reactor;
wherein said impregnating is controlled based on said flow of gases.

2. The method according to claim 1, wherein said impregnating comprises adding the sulfur dioxide and/or the sulfurous acid to the impregnation reactor in the form of an impregnation liquid comprising the sulfur dioxide absorbed in water and/or the sulfurous acid.

3. The method according to claim 2, further comprising:
controlling the hydrothermal treatment such that a temperature and a pressure in the pretreatment reactor is constant,
wherein said step of controlling the impregnating comprises:
if said flow increases, reducing the amount of impregnation liquid added to the impregnation reactor and/or decreasing the concentration of the impregnation liquid added to the impregnation reactor and/or decreasing a residence time in the impregnation reactor, and
if said flow decreases, increasing the amount of impregnation liquid added to the impregnation reactor and/or increasing the concentration of the impregnation liquid added to the impregnation reactor and/or increasing the residence time in the impregnation reactor.

4. The method according to claim 1, wherein said impregnating comprises adding sulfur dioxide in gaseous form.

5. The method according to claim 4, further comprising:
controlling the hydrothermal treatment such that a temperature and a pressure in the pretreatment reactor is constant,
wherein said step of controlling the impregnating comprises:
if said flow increases, decreasing a pressure in the impregnation reactor and/or decreasing a residence time in the impregnation reactor and/or decreasing venting from the impregnation reactor, and
if said flow decreases, increasing a pressure in the impregnation reactor and/or increasing a residence time in the impregnation reactor and/or increasing venting from the impregnation reactor.

6. The method according to claim 3, wherein said controlling the hydrothermal treatment such that temperature and sulfur dioxide partial pressure in the pretreatment reactor is constant comprises controlling an amount of steam fed to the pretreatment reactor and/or controlling a flow of gases vented off from an upper portion of the pretreatment reactor.

7. The method according to claim 1, wherein said determining said flow comprises determining a valve opening of a valve through which said gases vented off from an upper portion of the pretreatment reactor pass.

8. The method according to claim 1, wherein said determining said flow comprises using a flow meter.

9. The method according to claim 2, further comprising:
prior to said impregnating, compressing the biomass material,
wherein said impregnating comprises feeding the biomass material into an inlet of said impregnation reactor such that the biomass material expands into said impregnation reactor.

10. The method according to claim 9, wherein said adding comprises adding the impregnation liquid into the impregnation reactor such that the impregnation liquid is absorbed by the biomass material and/or such that an impregnation liquid level is formed in said impregnation reactor, which impregnation liquid level is below said inlet.

11. The method according to claim 9, wherein said impregnation reactor is at least partly filled with impregnation liquid up to a liquid level, wherein said feeding comprises feeding the biomass material being into said impregnation reactor at a lower level than said liquid level.

12. The method according to claim 10, wherein the impregnation reactor is arranged substantially vertically and/or at an acute angle relative a vertical direction, further comprising conveying the biomass material upwards in the impregnation reactor towards an outlet at an upper portion of the impregnation reactor where the biomass material is discharged and fed to the pretreatment reactor.

13. The method according to claim 2, wherein a concentration of the impregnation liquid is within an interval corresponding to 28-165 g sulfur dioxide/l.

14. The method according to claim 2, wherein impregnation liquid is added such that the mass of sulphur dioxide in the biomass material after impregnation is within an interval 1-100 kg SO₂/metric ton dry biomass material.

15. The method according to claim 2, wherein a temperature of the impregnation liquid is in the range 5-60° C.

16. The method according to claim 1, wherein a residence time in the impregnation reactor is 20 s-60 minutes.

17. A system for treating biomass material comprising:
an impregnation reactor configured to receive the biomass material, the impregnation reactor comprising an inlet configured to receive sulfur dioxide and/or sulfurous acid for impregnation of the biomass material;
a pretreatment reactor configured to receive impregnated biomass material from the impregnation reactor for hydrothermal treatment, the pretreatment reactor being a valve for determining a flow of gases emitted from an upper portion of the pretreatment reactor; and
a control system configured to control said impregnation reactor based on said flow of gases.

18. The system according to claim 17, wherein said control system is configured to control the addition of the sulfur dioxide and/or the sulfurous acid based on said flow of gases.

19. The system according to claim 17, wherein said impregnation reactor comprising a conveyer configured to convey the biomass material through the reactor and/or at least one controllable valve arranged at an upper portion of the impregnation reactor to allow gases to be vented off from the reactor, and wherein said control system is configured to control the addition of the sulfur dioxide and/or the sulfurous acid and/or said conveyer and/or said controllable valve based on said flow of gases.

20. The method according to claim 5, wherein said controlling the hydrothermal treatment such that temperature and sulfur dioxide partial pressure in the pretreatment reactor is constant comprises controlling an amount of steam fed to the pretreatment reactor and/or controlling a flow of gases vented off from an upper portion of the pretreatment reactor.

21. The method according to claim 11, wherein the impregnation reactor is arranged substantially vertically and/or at an acute angle relative a vertical direction, further comprising conveying the biomass material upwards in the impregnation reactor towards an outlet at an upper portion of the impregnation reactor where the biomass material is discharged and fed to the pretreatment reactor.
